SEQUENCE LISTING (17 sequences)

SEQ ID NO: 1 - DNA, 731 bp, Mus musculus
caggtcaaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctcagatggaggcggttcaggcggaggtggctctggcggtggcggatcggacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacaggcggccgc

SEQ ID NO: 2 - protein, 243 aa, Mus musculus
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 3 - protein, 243 aa, Mus musculus
QVKLQQSGAELVEPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDQVSLSCRASQSISDYLHWYQQKSHESPQLLIKYASQSISGIPSRFSGSGSGSDFTLSINSVEPEDVGVYYCQNGHSFPLTFGAGTELELEQAA

SEQ ID NO: 4 - DNA, 354 bp, Mus musculus
caggtccaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctca

SEQ ID NO: 5 - DNA, 321 bp, Mus musculus
gacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaa

SEQ ID NO: 6 - DNA, 45 bp, Artificial Sequence
  Linker used to produce an 8H9 scFV.
gatggaggcggttcaggcggaggtggctctggcggtggcggatcg

SEQ ID NO: 7 - protein, 118 aa, Mus musculus
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSS

SEQ ID NO: 8 - protein, 107 aa, Mus musculus
DIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGGGTKLELK

SEQ ID NO: 9 - protein, 15 aa, Artificial Sequence
  Linker used to produce an 8H9 scFV.
DGGGSGGGGSGGGGS

SEQ ID NO: 10 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 11 - protein, 4 aa, Pseudomonas aeruginosa
KDEL

SEQ ID NO: 12 - protein, 4 aa, Pseudomonas aeruginosa
REDL

SEQ ID NO: 13 - DNA, 42 bp, Artificial Sequence
  Oligonucleotide primer.
ctcgggacctccggaagctttcagctccagcttggtcccagc

SEQ ID NO: 14 - DNA, 54 bp, Artificial Sequence
  Oligonucleotide primer.
agctgctggatagtgcatatgcaggtccaactgcagcagtctggggctgaactg

SEQ ID NO: 15 - DNA, 93 bp, Artificial Sequence
  Oligonucleotide primer.
tgggtgaggcagaggcctgaacagtgtcttgagtggattggatggatttttgcctgaaccgcaagcttgtgaggagacggtgaccgtggtccc

SEQ ID NO: 16 - DNA, 48 bp, Artificial Sequence
  Oligonucleotide primer.
tctggcggtggccatatggacatcgagctcactcagtctccaaccacc

SEQ ID NO: 17 - DNA, 66 bp, Artificial Sequence
  Oligonucleotide primer.
ctcgggagaattctatcatttcagctccagcttggtcccacaaccgaacgtgagcggaaagctgtg

(19) United States
(12) Patent Application Publication
(10) Pub. No.: US 2006/0051359 A1
(43) Pub. Date: Mar. 9, 2006

(54) Recombinant immunotoxin and use in treating tumors

(76) Inventors: Ira Pastan, Potomac, MD (US); Masanori Onda, Rockville, MD (US); Nai-Kong Cheung, Purchase, NY (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/537,061

(22) PCT Filed: Dec. 1, 2003
(86) PCT No.: PCT/US03/38227

§ 371 Date: Jun. 1, 2005

Related U.S. Application Data
(63) Continuation-in-part of application No. 60/430,305, filed on Dec. 2, 2002.

(51) Int. Cl. A61K 39/00; C07K 14/82

(52) U.S. Cl. 424/185.1; 530/350

(57) ABSTRACT

Immunotoxins are disclosed that include a toxin, a variable region of a heavy chain of a monoclonal antibody that binds the antigen specifically bound by monoclonal antibody 8H9, and a variable region of a light chain of the monoclonal antibody that binds the antigen specifically bound by monoclonal antibody 8H9 and effector molecule. These immunotoxins include scFv and dsFv of monoclonal antibody 8H9. The immunotoxins are of use for the treatment of tumors.

### PRIORITY CLAIM

This application claims the benefit of U.S. Provisional Application No. 60/430,305, filed Dec. 2, 2003, which is incorporated herein by reference in its entirety.

### FIELD

This application relates to the field of immunotherapy, specifically to the use of antibodies and their use as immunotoxins for the treatment of cancer.

### BACKGROUND

Recombinant toxins are chimeric proteins in which a cell targeting moiety is fused to a toxin (Pastan et al., Science, 254:1173-1177, 1991). If the cell targeting moiety is the Fv portion of an antibody, the molecule is termed a recombinant immunotoxin (Chaudhary et al. Nature, 339:394-397, 1989). The toxin moiety is genetically altered so that it cannot bind to the toxin receptor present on most normal cells. Recombinant immunotoxins selectively kill cells which are recognized by the antigen binding domain. Fv fragments are the smallest functional modules of antibodies. When used to construct immunotoxins, Fv fragments are better therapeutic reagents than whole IgGs because their small size facilitates better tumor penetration (Yokota et al., Cancer Res., 52:3402-3408, 1992). Initially, Fvs were stabilized by making recombinant molecules in which the Variable Heavy (VH) and Variable Light (VL) domains are connected by a peptide linker so that the antigen binding domain site is regenerated in a single protein (a single chain Fv, or “scFv”) (Bird R., et al., Science, 242:423-426, 1988). Many Fvs, however, could not be stabilized by this approach.

An alternative approach is to stabilize the Fv by a disulfide bond that is engineered between framework regions of the two Fv domains. The disulfide-bond stabilized Fv (termed a “dsFv”) is fused to the toxin through either of the Fv domains (Brinkmann et al., Proc Natl Acad Sci USA, 90:7538-7542, 1993). These dsFv immunotoxins can often be produced very efficiently (Reiter et al., Biochem, 33:5451-5459, 1994).

During the past several years, a number of recombinant toxins have been made using different antibodies (“Abs”) (Reiter and Pastan, Trends Biotechnol., 16:513, 1998). Several of these recombinant immunotoxins have now been evaluated in phase I trials in patients with cancer, such as hematopoietic malignancies. However, there remains a need to develop additional antibodies that can be used to treat additional types of tumors.

### SUMMARY

The monoclonal antibody 8H9 binds the cells of many tumors, including sarcomas and carcinomas. However, this monoclonal antibody does not bind the cells of normal tissues. Fv fragments of this antibody have been produced which are capable of binding the epitopic determinant. Disclosed herein are immunotoxins including a toxin and an Fv fragment of monoclonal 8H9 which can be used to kill a tumor cell.

An isolated Fv protein is disclosed herein. The Fv protein includes a variable region of a heavy chain of a monoclonal antibody that binds the antigen specifically bound by monoclonal antibody 8H9 and a variable region of a light chain of the monoclonal antibody that binds the antigen specifically bound by monoclonal antibody 8H9. The Fv protein also includes an effector molecule that is a toxin. The Fv protein specifically binds the epitope bound by monoclonal antibody 8H9. In one example, the toxin is a Pseudomonas exotoxin.

In one example, the Fv protein is a single chain Fv. In another example, the Fv protein is a dsFv, wherein the variable region of a heavy chain of a monoclonal antibody that binds the antigen specifically bound by monoclonal antibody 8H9 and a variable region of a light chain of the monoclonal antibody that binds the antigen specifically bound by monoclonal antibody 8H9 are covalently linked by disulfide bonds.

Nucleic acids are disclosed that encode the Fv proteins. Vectors are also disclosed that include these nucleic acids, as are host cells including the vectors.

Methods are disclosed for using the isolated Fv proteins that specifically bind the antigen bound by monoclonal antibody 8H9. In one example, a method is disclosed for killing a tumor cell. The method includes contacting the tumor cell with a therapeutically effective amount of the isolated Fv protein.

Methods for treating a subject with a tumor are also disclosed. The method includes administering to the subject a therapeutically effective amount of a Fv protein that includes a variable region of a heavy chain of a monoclonal antibody that binds the antigen specifically bound by monoclonal antibody 8H9, a variable region of a light chain of the monoclonal antibody that binds the antigen specifically bound by monoclonal antibody 8H9, and a toxin.

The foregoing and other features and advantages will become more apparent from the following detailed description of several embodiments, which proceeds with reference to the accompanying figures.

### BRIEF DESCRIPTION OF THE FIGURES

FIGS. 1A-1B are digital images of PAGE of purified RITs. The purified proteins were run on 4-20% gradient SDS polyacrylamide electrophoresis gels. The gel was stained with Coomasie Blue. In FIG. 1A, Lane 1, 8H9(scFv)-PE38 (non-reduced). In FIG. 1B, Lane 1, 8H9(dsFv)-PE38 (reduced); Lane 2, 8H9(dsFv)-PE38 (non-reduced); M, molecular mass standards are (top to bottom) 250, 150, 100, 75, 50, 37, 25, 15, and 10 kDa, respectively.

FIG. 2 is a graph of cytotoxic activity of 8H9(scFv) ITs toward MCF-7 cell line. Cytotoxicity toward MCF-7 cells of 8H9(scFv)-PE38 (∘). M1(dsFv)-PE38 (□) was used as a negative control.

FIG. 3 is a graph of the specific cytotoxic activity of 8H9(scFv) ITs toward MCF-7 cell line. Competition cytotoxic activity of 8H9(scFv)-PE38 on MCF-7 cells by addition of excess 8H9 MAb (∘). MCF-7 cells (1.6×10⁴/well) were incubated with 15.5 ng/ml of 8H9(scFv)-PE38 and increasing concentrations of competing 8H9 MAb or control T6 MAb. Note that addition of equal amounts of control T6 MAb (□), which bind to a different antigen, does not compete.

FIG. 4 is a bar graph of the plasma level of 8H9(dsFv)-PE38 in monkeys. One Cynomolgus monkey was treated with 8H9(dsFv)-PE38 0.1 mg/kg QOD×3 (black bars), and a second monkey was treated with 0.2 mg/kg QOD×3 (white bars). Plasma was obtained on each of the 3 treatment days from each monkey 10 minutes after each dose. Vertical bars indicate the plasma levels. Error bars indicate standard deviations of the mean of triplicate cytotoxic activity experiments.

FIG. 5 is a line graph Anti-tumor effect of 8H9(Fv)-PE38 in SCID mice. Groups of animals were injected with 2×10⁶ MCF-7 cells (A, B, C, D) or OHS-M1 cells (E, F) on day 0. On day 4, tumors reached a size of 50 mm³. Animals were treated i.v. on days 4, 6, and 8 with 0.075 mg/kg (Δ), and 0.15 mg/kg (▴) 8H9 (scFv)-PE38 in Dulbecco's modified PBS containing 0.2% HSA or 0.075 (∘) and 0.15 (●) of 8H9(dsFv)-PE38 in DPBS (0.2% HSA). Control groups received diluent alone (▪), or M1(dsFv)-PE38 (□), which is a IT against CD25. No deaths were observed at these doses. Comparison of tumor size (*) between ▪ with Δ, ▴, or ∘ gives p<0.05. Data are expressed as the mean±SD (n=5 or 10).

FIG. 6 is a table (Table 4) showing the toxicity of 8H9(dsFv)-PE3 8 in monkeys.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:
SEQ ID NO: 1 is a nucleic acid sequence encoding an 8H9 scFv.
SEQ ID NO: 2 is an amino acid sequence of an 8H9 scFv.
SEQ ID NO: 3 is an amino acid sequence of an 8H9 scFv.
SEQ ID NO: 4 is a nucleic acid sequence encoding the V_{H} of 8H9.
SEQ ID NO: 5 is a nucleic acid sequence encoding the V_{L} of 8H9.
SEQ ID NO: 6 is a nucleic acid sequence of a linker.
SEQ ID NO: 7 is an amino acid sequence of a V_{H} of an antibody that binds the antigen specifically bound by monoclonal antibody 8H9.
SEQ ID NO: 8 is an amino acid sequence of the V_{L} of an antibody that binds the antigen specifically bound by monoclonal 8H9.
SEQ ID NO: 9 is the amino acid sequence of a linker.
SEQ ID NO: 10 is an amino acid sequence of Pseudomonas exotoxin.
SEQ ID NOS: 11-12 are amino acid sequences of segments of a Pseudomonas exotoxin.
SEQ ID NOS: 13-17 are the nucleic acid sequences of primers.

### DETAILED DESCRIPTION

I. Abbreviations

CDR: complementarity determining region

dsFv: disulfide stabilized fragment of a variable region

IT: immunotoxin

kDa: kilodaltons

LCDR: light chain complementarity determining region

HCDR: heavy chain complementarity determining region

QOD: every other day

PAGE: polyacrylamide gel electrophoresis

PE: Pseudomonas exotoxin

RIT: recombinant immunotoxin

s. c.: subcutaneous

SCID: severe combined immunodeficiency

scFv: single chain fragment of a variable region

SDS: sodium dodecyl sulphate

V_{H}: variable region of a heavy chain

V_{L}: variable region of a light chain

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Administration: The introduction of a composition into a subject by a chosen route. For example, if the chosen route is intravenous, the composition is administered by introducing the composition into a vein of the subject.

Amplification: Of a nucleic acid molecule (e.g., a DNA or RNA molecule) refers to use of a technique that increases the number of copies of a nucleic acid molecule in a specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Antibody: A polypeptide ligand comprising at least a light chain or heavy chain immunoglobulin variable region which specifically recognizes and binds an epitope (e.g., an antigen). This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, an immunoglobulin has a heavy and light chain. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat, E. et al., Sequences of Proteins of Immunological Interest, U.S. Department of Health and Human Services, 1991, which is hereby incorporated by reference. The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (e.g., see U.S. Pat. No. 5,585,089).

Breast cancer: A neoplastic condition of breast tissue that can be benign or malignant. The most common type of breast cancer is ductal carcinoma. Ductal carcinoma in situ is a non-invasive neoplastic condition of the ducts. Lobular carcinoma is not an invasive disease but is an indicator that a carcinoma may develop. Infiltrating (malignant) carcinoma of the breast can be divided into stages (I, IIA, IIB, IIIA, IIIB, and IV).

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating breast cancer, a sarcoma, a neuroblastoma, or another tumor. In one embodiment, a chemotherapeutic agent is a radioactive compound. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Combination chemotherapy is the administration of more than one agent to treat cancer. One example is the administration of an 8H9FV-PE38 used in combination with a radioactive or chemical compound.

Cytotoxicity: The toxicity of an immunotoxin to the cells intended to be targeted by the immunotoxin, as opposed to the cells of the rest of an organism. Unless otherwise noted, in contrast, the term “toxicity” refers to toxicity of an immunotoxin to cells others than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included in this disclosure as long as the amino acid sequence of the antibody or toxin molecule encoded by the nucleotide sequence is unchanged.

Effector molecule: A toxin that can be used to induce cytotoxicity. In one example, an effector molecule is a biological toxin, such as ricin, abrin, diphtheria toxin and subunits thereof, ribotoxin, ribonuclease, saporin, restrictocin, gelonin and calicheamicin, a Pseudomonas exotoxin, or botulinum toxins A through F. In another example, an effector molecule is not a radionucleotide.

Expression control sequence: A nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

A “promoter” is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

“Expression cassette” refers to a recombinant nucleic acid construct comprising an expression control sequence operatively linked to an expressible nucleotide sequence. An expression cassette generally comprises sufficient cis-acting elements for expression; other elements for expression can be supplied by the host cell or in vitro expression system.

“Expression vector” refers to a vector comprising an expression cassette. Expression vectors include all those known in the art, such as cosmids, plasmids (e.g., naked or contained in liposomes) and viruses that incorporate the expression cassette. An “expression plasmid” comprises a plasmid nucleotide sequence encoding a molecule or interest, which is operably linked to a promoter.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Inhibiting or treating a disease: Inhibiting the full development of a disease or condition, for example, in a subject who is at risk for a disease such as a tumor. “Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Linker: A molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

Linker peptide: A peptide that is used to join two protein sequences in an amino acid sequence. A linker can be included between an antibody binding fragment (e.g., Fv fragment) which serves to indirectly bond the variable domain of the heavy chain to the variable domain of the light chain.

Immunoconjugate or immunotoxin: A covalent linkage of an effector molecule to an antibody. Specific, non-limiting examples of toxins include, but are not limited to, abrin, ricin, Pseudomonas exotoxin (PE, such as PE35, PE37, PE38, and PE40), diphtheria toxin (DT), saporin, restrictocin, or modified toxins thereof, or other toxic agents that directly or indirectly inhibit cell growth or kill cells. For example, PE and DT are highly toxic compounds that typically bring about death through liver and heart toxicity in humans. PE and DT, however, can be modified into a form for use as an immunotoxin by removing the native targeting component of the toxin (e.g., domain Ia of PE and the B chain of DT) and replacing it with a different targeting moiety, such as an antibody. A “chimeric molecule” is a targeting moiety, such as a ligand or an antibody, conjugated (coupled) to an effector molecule. In one embodiment, an antibody is joined to an effector molecule (EM). In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule.

Monoclonal Antibody 8H9: A monoclonal antibody that binds the 8H9 antigen, which has a molecular weight of about 58 Kdaltons. The antibody is described, and the sequence of a scFv of monoclonal antibody 8H9 is set forth in PCT Publication No. 02/32375 A2 (see also published U.S. patent application No. US2003/10393A1 and published U.S. patent application No. US 2002/012264A1). All of these published patent applications are incorporated herein by reference. In one embodiment, the 8H9 heavy chain (H) sequence the Complementarity Determining Region (CDR)1 comprises an amino sequence NYDIN (amino acids 31-35 of SEQ ID NO: 3), the HCDR2 has an amino acid sequence WIFPGDGSTQY (amino acids 50-60 of SEQ ID NO: 3), the HCDR3 has an amino acid sequence QTTATWFAY (amino acids 99-107 of SEQ ID NO: 3). In addition, the light Complementarity Determining Region (LCDR1) has an amino acid sequence RASQSISDYLH (amino acids 157-167 of SEQ ID NO: 3), the LCDR2 has an amino acid sequence YASQSIS (amino acids 183-189 of SEQ ID NO: 3), and the LCDR3 has an amino acid sequence QNGHSFPLT (amino acids 222-230 of SEQ ID NO: 3). The term 8H9 also includes humanized forms of the antibody. The term “8H9 variable region” includes fragments of the antibody, such as single chain Fv (scFv) and disulfide stabilized Fv, and humanized forms of these fragments. An amino acid sequence an 8H9 heavy chain variable region (V_{H}) and an 8H9 light chain variable region (V_{L}) are set forth herein.

Naturally-occurring: As applied to an object, the term refers to the fact that the object can be found in nature. For example, an amino acid or nucleotide sequence that is present in an organism (including viruses) that can be isolated from a source in nature and which has not been intentionally modified by man in the laboratory is naturally-occurring.

Neoplasia and Tumor: The process of abnormal and uncontrolled growth of cells. Neoplasia is one example of a proliferative disorder.

The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant.” Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-0-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Pharmaceutical composition: A composition suitable for pharmaceutical (therapeutic) use in a mammal. A pharmaceutical composition comprises a therapeutically effective amount of an active agent and a pharmaceutically acceptable carrier.

“Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration). In one embodiment, a “pharmaceutically acceptable salt” is a salt that can be formulated into a compound for pharmaceutical use including, e.g., metal salts (sodium, potassium, magnesium, calcium, etc.) and salts of ammonia or organic amines.

Polypeptide or Protein: A polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional. A “peptide” refers to a polymer of amino acids of at most 20 amino acids in length, such as a polymer of eight, ten, twelve, fifteen or eighteen amino acids in length.

The term “residue” or “amino acid residue” or “amino acid” includes reference to an amino acid that is incorporated into a protein, polypeptide, or peptide. The amino acid can be a naturally occurring amino acid and, unless otherwise limited, can encompass known analogs of natural amino acids that can function in a similar manner as naturally occurring amino acids.

A “conservative substitution”, when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table A each contain amino acids that are conservative substitutions for one another:

### Table A

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   See also, Creighton, Proteins, W.H. Freeman and Company, New York, 1984.

For purposes of this application, amino acids are classified as acidic or basic, or as negatively or positively charged, depending on their usual charge at neutral pH (physiological pH is generally considered to be about 7.4). Lysine and arginine are basic amino acids which carry a positive charge at neutral pH. Aspartic acid and glutamic acid are acidic amino acids that carry a negative charge at neutral pH. Three other amino acids, histidine (which can be uncharged or positively charged depending on the local environment), cysteine, and tyrosine, have readily ionizable side chains, see generally, Stryer, L. Biochemistry, W. H. Freeman and Co., New York (4^{th} Ed., 1995); however, cysteine and tyrosine are only positively charged at higher pH and are not considered basic residues for purposes of the methods taught herein.

The term “substantially similar” in the context of a peptide indicates that a peptide comprises a sequence with at least 90%, preferably at least 95% sequence identity to the reference sequence over a comparison window of 10-20 amino acids. The percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. The cysteines which form the disulfide bond are within the framework regions of the single chain antibody and serve to stabilize the conformation of the antibody.

The terms “conjugating,” “joining,” “bonding” or “linking” refer to making two polypeptides into one contiguous polypeptide molecule. The terms include reference to joining an antibody moiety to an effector molecule (EM), and to joining a heavy chain variable region with a light chain variable region. The linkage can be either by chemical or recombinant means. Chemical means refers to a reaction between the two proteins such that there is a covalent bond formed between the two molecules to form one molecule.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, underexpressed or not expressed at all.

Selectively reactive or specific binding: The preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10-fold and most preferably greater than 100-fold increase in amount of bound antibody (per unit time) to a cell or tissue bearing the antigen recognized by 8H9 as compared to a cell or tissue lacking expression of the antigen. Specific binding to a protein under such conditions requires an antibody that is selected for its specificity for a particular protein. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay formats and conditions. Preferably, the immunologically reactive conditions employed in the methods disclosed herein are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The term “contacting” includes reference to placement in direct physical association.

Single chain Fv or “scFv”: An antibody in which the variable regions of the heavy chain and of the light chain of a traditional two chain antibody have been joined to form one chain. Typically, a linker peptide is inserted between the two chains to allow for proper folding and creation of an active binding site.

Stringent hybridization conditions: Conditions in which a nucleic acid sequence selectively hybridizes to its corresponding antisense nucleic acid sequence, and not to unrelated nucleic acid sequences. One example of stringent hybridization conditions is 50% formamide, 5×SSC and 1% SDS incubated at 42° C. or 5×SSC and 1% SDS incubated at 65° C., with a wash in 0.2×SSC and 0.1% SDS at 65° C.

Subject: Any human or non-human mammal.

Substantially pure or isolated: A composition in which an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

Targeting moiety: The portion of an immunoconjugate, such as an immunotoxin, intended to target the immunoconjugate to a cell of interest. Typically, the targeting moiety is an antibody, a scFv, a dsFv, an Fab, or an F(ab′)2.

Toxic moiety: The portion of an immunotoxin which renders the immunotoxin cytotoxic to cells of interest.

Therapeutically effective amount: A dosage of a therapeutic agent sufficient to produce a desired result. In one example, a therapeutically effective amount is the amount of an immunotoxin sufficient to inhibit cell protein synthesis by at least 50%. In another example, a therapeutically effective amount is an amount sufficient to kill a target cell.

Toxin: A molecule that is cytotoxic for a cell. Toxins include abrin, ricin, Pseudomonas exotoxin (PE), diphtheria toxin (DT), botulinum toxin, saporin, restrictocin or gelonin or modified toxins thereof. For example, PE and DT are highly toxic compounds that typically bring about death through liver toxicity. PE and DT, however, can be modified into a form for use as an immunotoxin by removing the native targeting component of the toxin (e.g., domain Ia of PE or the B chain of DT) and replacing it with a different targeting moiety, such as an antibody.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Antibodies and Immunotoxins

Immunotoxins including a toxin and an 8H9 variable region are disclosed herein. The 8H9 monoclonal antibody and scFvs of the 8H9 monoclonal antibody have been described previously (see published U.S. patent application No. US2003/10393A1 and PCT Publication No. 02/32375 A2, both of which are incorporated herein by reference).

In one embodiment, an 8H9 scFv is encoded by the nucleic acid sequence

(SEQ ID NO: 1)

CAGGTCAAACTGCAGCAGTCTGGGGCTGAACTGGTAAAGCCTGG
GGCTTCAGTGAAATTGTCCTGCAAGGCTTCTGGCTACACCTTCAC
AAACTATGATATAAACTGGGTGAGGCAGAGGCCTGAACAGGGAC
TTGAGTGGATTGGATGGATTTTTCCTGGAGATGGTAGTACTCAAT
ACAATGAGAAGTTCAAGGGCAAGGCCACACTGACTACAGACACA
TCCTCCAGCACAGCCTACATGCAGCTCAGCAGGCTGACATCTGAG
GACTCTGCTGTCTATTTCTGTGCAAGACAGACTACGGCTACCTGGT
TTGCTTACTGGGGCCAAGGGACCACGGTCACCGTGTCCTCAGATG
GAGGCGGTTCAGGCGGAGGTGGCTCTGGCGGTGGCGGATGGGAC
ATCGAGCTCACTCAGTCTCCAACCACCCTGTCTGTGACTCCAGGA
GATAGAGTCTCTCTTTCCTGCAGGGCCAGCCAGAGTATTAGCGAG
TACTTACACTGGTACCAACAAAAATCACATGAGTCTCCAAGGCTT
CTCATCAAATATGCTTCCCAATCCATCTCTGGGATCCCCTCCAGGT
TCAGTGGCAGTGGATCAGGGTCAGATTTCACTCTCAGTATCAACA
GTGTGGAACCTGAAGATGTTGGAGTGTATTACTGTCAAAATGGTC
ACAGCTTTCCGCTCACGTTCGGTGCTGGGACCAAGCTGGAGCTGA
AACAGGCGGCCGC

In a further embodiment, an 8H9 scFv has an amino acid sequence set forth as:

(SEQ ID NO: 2)

QVKLQQSGAELVKPGASVKLSCKASGYTFTNYDINWVRQRPEQGLE
WIGWIFPGDGSTQYNEKFKGKATLTTDTSSSTAYMQLSRLTSEDSAV
YFCARQTTATWFAYWGQGTTVTVSSDGGGSGGGGSGGGGSDIELTQ
SPTTLSVTPGDRVSLSCRASQSISDYLHWYQQKSHESPRLLIKYASQSI
SGIPSRFSGSGSGSDFTLSINSVEPEDVGVYYCQNGHSFPLTFGAGTKL
ELKQAA

In an additional embodiment, an 8H9 scFv has an amino acid sequence set forth as:

(SEQ ID NO: 3)

QVKLQQSGAELVEPGASVKLSCKASGYTFTNYDINWVRQRPEQGLE
WIGWIFPGDGSTQYNEKFKGKATLTTDTSSSTAYMQLSRLTSEDSAV
YFCARQTTATWFAYWGQGTTVTVSSDGGGSGGGGSGGGGSDIELTQ
SPTTLSVTPGDQVSLSCRASQSISDYLHWYQQKSHESPQLLIKYASQSI
SGIPSRFSGSGSGSDFTLSINSVEPEDVGVYYCQNGHSFPLTFGAGTEL
ELEQAA

In a further embodiment, an 8H9 antibody includes a heavy chain variable region (V_{H}) encoded by a nucleic acid sequence set forth as:

(SEQ ID NO:4)

CAG GTC CAA CTG CAG CAG TCT GGG GCT GAA CTG GTA
AAG CCT GGG GCT TCA GTG AAA TTG TCC TGC AAG GCT
TCT GGC TAC ACC TTC ACA AAC TAT GAT ATA AAC TGG
GTG AGG CAG AGG CCT GAA CAG GGA CTT GAG TGG ATT
GGA TGG ATT TTT CCT GGA GAT GGT AGT ACT CAA TAC
AAT GAG AAG TTC AAG GGC AAG GCC ACA CTG ACT ACA
GAC ACA TCC TCC AGC ACA GCC TAC ATG CAG CTC AGC
AGG CTG ACA TCT GAG GAG TCT GCT GTC TAT TTC TGT
GCA AGA CAG ACT ACG GCT ACC TGG TTT GCT TAC TGG
GGC CAA GGG ACC ACG GTC ACC GTC TCC TCA,

and a light chain variable region (V_{L}) encoded by a nucleic acid set forth as

(SEQ ID NO: 5)

GAC ATC GAG CTC ACT CAG TCT CCA ACC ACC CTG TCT
GTG ACT CCA GGA GAT AGA GTC TCT CTT TCC TGC AGG
GCC AGC CAG AGT ATT AGC GAC TAC TTA CAC TGG TAC
CAA CAA AAA TCA CAT GAG TCT CCA AGG CTT CTC ATC
AAA TAT GCT TCC CAA TCC ATC TCT GGG ATC CCC TCC
AGG TTC AGT GGC AGT GGA TCA GGG TCA GAT TTC ACT
CTC AGT ATC AAC AGT GTG GAA CCT GAA GAT GTT GGA
GTG TAT TAC TGT CAA AAT GGT CAC AGC TTT CCG CTC
ACG TTC GGT GCT GGG ACC AAG CTG GAG CTG AAA.

These two nucleic acid sequences (SEQ ID NO:4 and SEQ ID NO:5), can be used to produce an 8H9 scFv by inserting a linker between the two nucleic acid sequences.

In one example, a suitable linker has an nucleic acid sequence set forth as:

(SEQ ID NO: 6)

GAT GGA GGC GGT TCA GGC GGA GGT GGC TCT GGC GGT
GGC GGA TCG

In one embodiment, an 8H9 antibody has a V_{H} including an amino acid sequence set forth as:

(SEQ ID NO:7)

QVQLQQSGAELVKPGASVKLSCKASGYTFTNYDINWVRQRPEQGLE
WIGWIFPGDGSTQYNEKFKGKATLTTDTSSSTAYMQLSRLTSEDSAV
YFCARQTTATWFAYWGQGTTVTVSS,

and a V_{L} including an amino acid sequence set forth as:

DIELTQSPTTLSVTPGDRVSLSCRASQSISDYLHWYQQKSHESPRLLIK
YASQSISGIPSRFSGSGSGSDFTLSINSVEPEDVGVYYCQNGHSFPLTF
GGGTKLELK.

These two amino acid sequences (SEQ ID NO:7 and SEQ ID NO: 8 can be used to produce an 8H9 scFv by inserting a linker between the two amino acid sequences.

In one example, a suitable linker has an amino acid sequence set forth as:

| | |
|---|---|
| DGGGSGGGGSGGGGS | (SEQ ID NO:9) |

In a further embodiment, an 8H9 antibody includes the heavy (H) and light (L) chain Complementarity Determining Regions (CDR) of 8H9. Heavy chain Complementarity Determining Region (HCDR)1 comprises an amino sequence NYDIN (amino acids 31-35 of SEQ ID NO: 3 or 7), the HCDR2 has an amino acid sequence WIFPGDGSTQY (amino acids 50-60 of SEQ ID NO: 3 or SEQ ID NO: 7), the HCDR3 has an amino acid sequence QTTATWFAY (amino acids 99-107 of SEQ ID NO: 3 or 7). In addition, the light Complementarity Determining Region (LCDR1) has an amino acid sequence RASQSISDYLH (amino acids 157-167 of SEQ ID NO: 3 or amino acids 24-34 of SEQ ID NO: 8), the LCDR2 has an amino acid sequence YASQSIS (amino acids 183-189 of SEQ ID NO: 3, amino acids 50-56 of SEQ ID NO: 8), and the LCDR3 has an amino acid sequence QNGHSFPLT (amino acids 222-230 of SEQ ID NO: 3, amino acids 89-97 of SEQ ID NO: 8).

The antibody or antibody fragment can be a humanized immunoglobulin having complementarity determining regions (CDRs) from a donor 8H9 immunoglobulin and heavy and light chain variable region frameworks from human acceptor immunoglobulin heavy and light chain frameworks. Generally, the humanized immunoglobulin specifically binds to the epitope bound by the 8H9 antibody with an affinity constant of at least 10⁷ M⁻¹, such as at least 10⁸ M⁻¹ or 10⁹ M⁻¹.

Humanized monoclonal antibodies are produced by transferring donor (8H9) complementarity determining regions from heavy and light variable chains of the mouse immunoglobulin into a human variable domain, and then substituting human residues in the framework regions of the donor counterparts. The use of antibody components derived from humanized monoclonal antibodies obviates potential problems associated with the immunogenicity of the constant regions of the donor antibody. Techniques for producing humanized monoclonal antibodies are described, for example, by Jones, et al., Nature 321:522, 1986; Riechmann, et al., Nature 332:323, 1988; Verhoeyen, et al., Science 239:1534, 1988; Carter, et al., Proc. Nat'l Acad Sci. USA. 89:4285, 1992; Sandhu, Crit. Rev. Biotech. 12:437, 1992; and Singer, et al., J. Immunol.150:2844, 1993.

In one embodiment, the sequence of the humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in a humanized antibody framework regions, are known in the art (see, for example, in U.S. Pat. No. 5,585,089).

Antibodies include intact molecules as well as fragments thereof, such as Fab, F(ab′)₂, and Fv which include a heavy chain and light chain variable region and are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the variable region of the light chain and the variable region of the heavy chain expressed as two chains; and

(5) Single chain antibody (such as scFv), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

In one example, the variable region included in the immunotoxin is an 8H9 Fv, which includes the variable region of the light chain and the variable region of the heavy chain expressed as individual polypeptides. In one group of embodiments, the antibodies have V_{L} and V_{H} regions having the amino acid sequence shown above (for example, see SEQ ID NO: 7 and SEQ ID NO:8). Fv antibodies are typically about 25 kDa and contain a complete antigen-binding site with 3 CDRs per each heavy chain and each light chain. The V_{H} and the V_{L} can be expressed from two individual nucleic acid constructs. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

One of skill will realize that conservative variants of the antibodies can be produced. Such conservative variants employed in dsFv fragments or in scFv fragments will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield.

Antibody fragments can be prepared by proteolytic hydrolysis of the antibody or by expression in E. coil of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff, et al., Arch. Biochem. Biophys. 89:230, 1960; Porter, Biochem. J. 73:119, 1959; Edelman, et al., Methods in Enzymology, Vol. 1, page 422, Academic Press, 1967; and Coligan, et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar, et al., Proc. Nat'l Acad. Sci. USA 69:2659, 1972). Alternatively, the variable chains can be linked by an intermolecular disulfide bond or cross-linked by chemicals such as glutaraldehyde. See, e.g., Sandhu, supra. Thus, a dsFv can be produced. In an additional example, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow, et al., Methods: a Companion to Methods in Enzymology, Vol. 2, page 97, 1991; Bird, et al., Science 242:423, 1988; U.S. Pat. No. 4,946,778; Pack, et al., Bio/Technology 11:1271, 1993; and Sandhu, supra).

Immunoconjugates include, but are not limited to, molecules in which there is a covalent linkage of a therapeutic agent with an antibody. A therapeutic agent is an agent with a particular biological activity directed against a particular target molecule or a cell bearing a target molecule. Therapeutic agents include various drugs such as vinblastine, daunomycin and the like, and effector molecules such as cytotoxins such as native or modified Pseudomonas exotoxin or Diphtheria toxin, encapsulating agents, (e.g., liposomes) which themselves contain pharmacological compositions, target moieties and ligands.

The choice of a particular therapeutic agent depends on the particular target molecule or cell and the biological effect is desired to evoke. Thus, for example, the therapeutic agent may be an effector molecule that is cytotoxin which is used to bring about the death of a particular target cell. Conversely, where it is merely desired to invoke a non-lethal biological response, a therapeutic agent can be conjugated to a non-lethal pharmacological agent or a liposome containing a non-lethal pharmacological agent.

Toxins can be employed with 8H9 antibodies and 8H9 fragments, such as an 8H9 svFv or a dsFv, to yield chimeric molecules, which are of use as immunotoxins. Exemplary toxins include Pseudomonas exotoxin (PE), ricin, abrin, diphtheria toxin and subunits thereof, ribotoxin, ribonuclease, saporin, and calicheamicin, as well as botulinum toxins A through F. These toxins are well known in the art and many are readily available from commercial sources (for example, Sigma Chemical Company, St. Louis, Mo.).

Diphtheria toxin is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. ViroL 19:220, 1976), and has been used in human clinical trials. See, U.S. Pat. No. 5,792,458 and U.S. Pat. No. 5,208,021. As used herein, the term “diphtheria toxin” refers as appropriate to native diphtheria toxin or to diphtheria toxin that retains enzymatic activity but which has been modified to reduce non-specific toxicity.

Ricin is the lectin RCA60 from Ricinus communis (Castor bean). The term “ricin” also references toxic variants thereof. For example, see, U.S. Pat. No. 5,079,163 and U.S. Pat. No. 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543, 1972). The A chain is responsible for inactivating protein synthesis and killing cells. The B chain binds ricin to cell-surface galactose residues and facilitates transport of the A chain into the cytosol (Olsnes et al., Nature 249:627-631, 1974 and U.S. Pat. No. 3,060,165).

Ribonucleases have also been conjugated to targeting molecules for use as immunotoxins (see Suzuki et al., Nat Biotech 17:265-70, 1999). Exemplary ribotoxins such as α-sarcin and restrictocin are discussed in, e.g., Rathore et al., Gene 190:31-5, 1997; and Goyal and Batra, Biochem 345 Pt 2:247-54, 2000. Calicheamicins were first isolated from Micromonospora echinospora and are members of the enediyne antitumor antibiotic family that cause double strand breaks in DNA that lead to apoptosis (see, e.g., Lee et al., J. Antibiot 42:1070-87. 1989). The drug is the toxic moiety of an immunotoxin in clinical trials (see, e.g., Gillespie et al., Ann Oncol 11:735-41, 2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al., Agr. Biol. Chem. 52:1095, 1988; and Olsnes, Methods Enzymol. 50:330-335, 1978).

In one embodiment, the toxin is Pseudomonas exotoxin (PE). Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence and the sequence of modified PE is provided in U.S. Pat. No. 5,602,095, incorporated herein by reference. In one embodiment, native PE has a sequence set forth as:

| (SEQ ID NO: 10) | |
|---|---|
| | AEEAFDLWNE CAKACVLDLK DGVRSSRMSV DPAIADTNGQ GVLHYSMVLE |
| | GGNDALKLAI DNALSITSDG LTIRLEGGVE PNKPVRYSYT RQARGSWSLN |
| | WLVPIGHEKP SNIKVFIHEL NAGNQLSHMS PIYTIEMGDE LLAKLARDAT |
| | FFVRAHESNE MQPTLAISHA GVSVVMAQTQ PRREKRWSEW ASGKVLCLLD |
| | PLDGVYNYLA QQRCNLDDTW EGKIYRVLAG NPAKHDLDIK PTVISHRLHF |
| | PEGGSLAALT AHQACHLPLE TFTRHRQPRG WEQLEQCGYP VQRLVALYLA |
| | ARLSWNQVDQ VIRNALASPG SGGDLGEAIR EQPEQARLAL TLAAAESERF |
| | VRQGTGNDEA GAANADVVSL TCPVAAGECA GPADSGDALL ERNYPTGAEF |
| | LGDGGDVSFS TRGTQNWTVE RLLQAHRQLE ERGYVFVGYH GTFLEAAQSI |
| | VFGGVRARSQ DLDAIWRGFY IAGDPALAYG YAQDQEPDAR GRIRNGALLR |
| | VYVPRSSLPG FYRTSLTLAA PEAAGEVERL IGHPLPLRLD AITGPEEEGG |
| | RLETILGWPL AERTVVIPSA IPTDPRNVGG DLDPSSIPDK EQAISALPDY |
| | ASQPGKPPRE DLK |

The method of action of PE is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall et al., J. Biol. Chem. 264:14256-14261, 1989.

The term “Pseudomonas exotoxin” (“PE”) as used herein refers as appropriate to a full-length native (naturally occurring) PE or to a PE that has been modified. Such modifications may include, but are not limited to, elimination of domain Ia, various amino acid deletions in domains Ib, II and III, single amino acid substitutions and the addition of one or more sequences at the carboxyl terminus, such as KDEL (SEQ ID NO:11) and REDL (SEQ ID NO:12). See Siegall et al., supra In several examples, the cytotoxic fragment of PE retains at least 50%, preferably 75%, more preferably at least 90%, and most preferably 95% of the cytotoxicity of native PE. In one embodiment, the cytotoxic fragment is more toxic than native PE.

Thus, the PE used in the immunotoxins disclosed herein includes the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE known in the art include PE40, PE38, and PE35.

In several embodiments, the PE has been modified to reduce or eliminate non-specific cell binding, typically by deleting domain Ia, as taught in U.S. Pat. No. 4,892,827, although this can also be achieved, for example, by mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”) exhibits greatly diminished non-specific cytotoxicity. This mutant form of PE is sometimes referred to as PE4E.

PE40 is a truncated derivative of PE (see, Pai et al., Proc. Nat'l Acad. Sci. USA 88:3358-62, 1991; and Kondo et al., J. Biol. Chem. 263:9470-9475, 1988). PE35 is a 35 kD carboxyl-terminal fragment of PE in which amino acid residues 1-279 have deleted and the molecule commences with a met at position 280 followed by amino acids 281-364 and 381-613 of native PE. PE35 and PE40 are disclosed, for example, in U.S. Pat. No. 5,602,095 and U.S. Pat. No. 4,892,827.

In some embodiments, the cytotoxic fragment PE38 is employed. PE38 is a truncated PE pro-protein composed of amino acids 253-364 and 381-613 of SEQ ID NO: 4 which is activated to its cytotoxic form upon processing within a cell (see e.g., U.S. Pat. No. 5,608,039, and Pastan et al., Biochim. Biophys. Acta 1333:C1-C6, 1997).

While in some embodiments, the PE is PE4E, PE40, or PE38, any form of PE in which non-specific cytotoxicity has been eliminated or reduced to levels in which significant toxicity to non-targeted cells does not occur can be used in the immunotoxins disclosed herein so long as it remains capable of translocation and EF-2 ribosylation in a targeted cell.

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

With the antibodies and immunotoxins herein provided, one of skill can readily construct a variety of clones containing functionally equivalent nucleic acids, such as nucleic acids which differ in sequence but which encode the same effector molecule (“EM”) or antibody sequence. Thus, nucleic acids encoding antibodies and conjugates and fusion proteins are provided herein.

Nucleic acid sequences encoding the immunotoxins can be prepared by any suitable method including, for example, cloning of appropriate sequences or by direct chemical synthesis by methods such as the phosphotriester method of Narang, et al., Meth. Enzymol. 68:90-99, 1979; the phosphodiester method of Brown, et al., Meth. Enzymol. 68:109-151, 1979; the diethylphosphoramidite method of Beaucage, et al., Tetra. Lett. 22:1859-1862, 1981; the solid phase phosphoramidite triester method described by Beaucage & Caruthers, Tetra Letts. 22(20):1859-1862, 1981, e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In one embodiment, the nucleic acid sequences encoding the immunotoxin are prepared by cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA chemical company (Saint Louis, Mo.), R&D systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In one example, an immunotoxin of use is prepared by inserting the cDNA which encodes an 8H9 variable region into a vector which comprises the cDNA encoding the EM. The insertion is made so that the variable region and the EM are read in frame so that one continuous polypeptide is produced. The polypeptide contains a functional Fv region and a functional EM region. In one embodiment, cDNA encoding a cytotoxin is ligated to a scFv so that the cytotoxin is located at the carboxyl terminus of the scFv. In one example, cDNA encoding a Pseudomonas exotoxin (“PE”), mutated to eliminate or to reduce non-specific binding, is ligated to a scFv so that the toxin is located at the amino terminus of the scFv. In another example, PE38 is located at the amino terminus of the 8H9 scFv. In a further example, cDNA encoding a cytotoxin is ligated to a heavy chain variable region of an antibody that binds the antigen specifically bound by 8H9, so that the cytoxin is located at the carboxyl terminus of the heavy chain variable region. The heavy chain-variable region can subsequently be ligated to a light chain variable region of the antibody that specifically binds 8H9 using disulfide bonds. In a yet another example, cDNA encoding a cytotoxin is ligated to a light chain variable region of an antibody that binds the antigen specifically bound by 8H9, so that the cytotoxin is located at the carboxyl terminus of the light chain variable region. The light chain-variable region can subsequently be ligated to a heavy chain variable region of the antibody that specifically binds 8H9 using disulfide bonds.

Once the nucleic acids encoding the 8H9 immunotoxin is isolated and cloned, the protein can be expressed in a recombinantly engineered cell such as bacteria, plant, yeast, insect and mammalian cells. One or more DNA sequences encoding 8H9 immunotoxin can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding the immunotoxin can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding the immunotoxin can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the immunotoxin, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Isolation and purification of recombinantly expressed polypeptide may be carried out by conventional means including preparative chromatography and immunological separations. Once expressed, the recombinant immunotoxins can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, incorporated by reference herein, and especially as described by Buchner et al., supra.

Renaturation is typically accomplished by dilution (e.g., 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

In addition to recombinant methods, the immunoconjugates, EM, and antibodies disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed. , Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N, N′-dicycylohexylcarbodiimide) are well known in the art.

### Pharmaceutical Compositions and Therapeutic Methods

Compositions are provided herein that include an immunotoxin that specifically binds the antigen bound by monoclonal antibody 8H9 and a pharmaceutically acceptable carrier. The compositions can be prepared in unit dosage forms for administration to a subject. The amount and timing of administration are at the discretion of the treating physician to achieve the desired purposes. In one example, the immunotoxin is formulated for parenteral administration, such as intravenous administration. In other examples, the immunotoxin is formulated for systemic or local (such as intra-tumor) administration.

The compositions for administration will commonly comprise a solution of the immunotoxin dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

A typical pharmaceutical immunotoxin composition for intravenous administration includes about 0.1 to 10 mg per patient per day. Dosages from 0.1 up to about 100 mg per patient per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed, Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% Sodium Chloride, USP, and typically administered at a dosage of from 0.5 to 15 mg/kg of body weight. Considerable experience is available in the art in the administration of antibody drugs, which have been marketed in the U.S. since the approval of Rituxan® in 1997. Antibody drugs can be administered by slow infusion, rather than in an IV push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The immunotoxins can be administered to slow or inhibit the growth of cells of that express the antigen specifically bound by 8H9, such as tumor cells. In these applications, a therapeutically effective amount of an immunotoxin that binds the antigen is administered to a subject in an amount sufficient to inhibit growth of antigen-expressing cells. Suitable subjects include those with a tumor that express the antigen bound by monoclonal antibody 8H9. Thus, suitable subjects include subjects that have a desmoplastic small round cell tumor, a brain tumor, a childhood sarcoma, neuroblastoma, and adenocarcinomas.

For example, the subject can have breast cancer, a globlastoma, a mixed glioma, an aligodendrogliomas, an astrocytoma, a meningiomas, a schwannomas, a medullobalstoma, a neurofibroma, a neuronoglial tumor, an ependymoma, a pineoblastoma, a Ewing's primitive neuroectodermal tumor, a rhabdomyosarcoma, an osteosarcoma, a synovial sarcoma, a leiomyosarcoma, a malignant fibrous histiocytoma, a neuroblastoma, a melanoma, a hepatoblastoma, a Wilm's tumor, or a rhabdoid tumor (see Modak et al., Cancer Res. 61: 404-84054, 2001). Amounts effective for this use will depend upon the severity of the disease and the general state of the patient's health. A therapeutically effective amount of the immunotoxin is that which provides either subjective relief of a symptom(s) or an objectively identifiable improvement as noted by the clinician or other qualified observer. These compositions can be administered in conjunction with another chemotherapeutic agent, either simultaneously or sequentially.

Single or multiple administrations of the compositions are administered depending on the dosage and frequency as required and tolerated by the patient. In any event, the composition should provide a sufficient quantity of the immunotoxins or antibodies disclosed herein to effectively treat the patient. The dosage can be administered once but may be applied periodically until either a therapeutic result is achieved or until side effects warrant discontinuation of therapy. In one example, a dose of the immunotoxin is infused for thirty minutes every other day. In this example, about one to about ten doses can be administered, such as three or six doses can be administered every other day. In a further example, a continuous infusion is administered for about five to about ten days. The subject can be treated with the immunotoxin at regular intervals, such as monthly, until a desired therapeutic result is achieved. Generally, the dose is sufficient to treat or ameliorate symptoms or signs of disease without producing unacceptable toxicity to the patient.

Controlled release parenteral formulations of the immunoconjugate compositions of the immunotoxin can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992) both of which are incorporated herein by reference.

Polymers can be used for ion-controlled release of immunoconjugate compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425-434, 1992; and Pec et al., J. Parent. Sci. Tech 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known. See, e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496, each of which is incorporated herein by reference.

Among various uses of the immunotoxins are included a variety of disease conditions caused by specific human cells that may be eliminated by the toxic action of the fusion protein, such as the treatment of malignant cells expressing the antigen specifically bound by monoclonal antibody 8H9.

The disclosure is illustrated by the following non-limiting Examples.

### EXAMPLES

An immunotoxin containing an 8H9 single chain Fv (scFv) and a toxin has been constructed. It is demonstrated herein that 8H9(scFv)-PE38 selectively kills cells that react with the MAb 8H9. Administration of the immunotoxin produces regressions of two human cancers growing in SCID mice that express the 8H9 antigen. A disulfide linked Fv (dsFv) immunotoxin has also been produced. This dsFv is suitable for clinical development as it is stable and is produced in a high yield during refolding and purification. The experiments described herein demonstrate that 8H9(dsFv)-PE38 is cytotoxic to MCF-7 cells, produces tumor regressions in nude mice, and is well tolerated by monkeys. Thus, both the 8H9 scFv and the dsFv can be used to kill tumor cells, and to reduce tumor burden.

### Example 1

### Materials and Methods

MAb 8H9 is a murine IgGl derived from the fusion of mouse myeloma SP2/0 cells and splenic lymphocytes from BALB/c mice immunized with a human neuroblastoma. Using immunohistochemistry MAb 8H9 was shown to be highly reactive with human brain tumors, childhood sarcomas, and neuroblastomas. In contrast, 8H9 is not reactive with normal human tissues. Immunofluorescence studies show that the 8H9 antigen is present on the external surface of tumor cell membranes. The antigen is not yet fully characterized but has the properties of a glycoprotein (10). As demonstrated herein, the presence of the antigen on the surface of cancer cells makes it a useful target for immunotoxin therapy.

Cell Lines: Human neuroblastoma cell lines were provided by Dr. Robert Seeger (LA-N-1), Children's Hospital of Los Angeles, Los Angeles, Calif. and by Dr. Shuen-Kuei Liao (NMB-7), McMaster University, Ontario, Canada. Cell lines were cultured in 10% fetal bovine serum in RPM1 1640 medium with L-glutamine, penicillin, and streptomycin. The human osteosarcoma cell line, OHS was established at the Norwegian Radium Hospital. It was maintained for several passages in DMEM supplemented with 10% fetal bovine serum and penicillin-streptomycin. OHS-M1 is a subline of OHS, isolated from a tumor growing subcutaneously in SCID mice. L428 (from Dr. C. S. Duckett, National Institutes of Health, Bethesda, Md.) is a Hodgkin's lymphoma cell line.

Construction of Plasmid for Expressions of Immunotoxin (IT): DNA encoding the 8H9 Fv in a single chain form was previously described (Cheung et al., Hybrid. Hybridomic, 21:433-443, 2002). Primers were designed to clone the DNA fragment encoding the 8H9 Fv into the PE38 expression vector. The V_{H} 5′ primer introduced an Nde I restriction site (underlined) and the V_{L} 3′ primer a Hind III restriction site (underlined) to facilitate cloning of the single chain antibody variable domain (scFv) into the expression vector. Because the cloned Fv contained an uncommon residue at position 3(K) (Kabat number) in the V_{H}, Fv was designed and produced as follows. Lysine at position 3 of the V_{H}, was substituted with Q. The following primers were used for making the scFv;

(SEQ ID NO: 13)

VL3′, 5′- CTC ggg ACC TCC ggA AgC TTT CAg CTC CAg
CTT ggT CCC AgC -3′;

(SEQ ID NO: 14)

VH5′K3Q, 5′-AgC TgC Tgg ATA gTg CAT ATg CAg gTC
CAA CTg CAg CAg TCT ggg gCT gAA CTg-3′.

PCR fragments were digested with Nde I and Hind III restriction enzyme and cloned into the Nde I-Hind III site in the expression vector (Brinkman et al., Proc. Natl. Acad. Sci. USA 88: 8616-8620, 1991). Concerning the making of dsFv, the positions of disulfides for the stabilization of B3(Fv) were ??ORIGINALLY identified using computer-modeled structure of B3(Fv), generated by mutating and energy minimizing the amino acid sequence and structure of McPC603, as described previously (Brinkman et al., Proc. Natl. Acad. Sci. USA 90:7538-7542, 1993). The amino acid sequences of 8H9(Fv) was simply aligned with that of B3(Fv) to determine the positions to insert cysteine residues. For the construction of 8H9(dsFv) fragments, cysteine residues were introduced in the V_{H} and V_{L} using PCR as previously described (Reiter et al., Biochemistry 33: 5451-5459, 1994). The following primers were used for making the dsFv;

(SEQ ID NO: 15)

```
STUVH,5′- Tgg gTg Agg CAg Agg CCT gAA CAg TgT CTT
        gAg Tgg ATT ggA Tgg ATT TTT -3′; HinH, 5′- gCC
        TgA ACC gCA AgC TTg TgA ggA gAC ggT gAC CgT
        ggT CCC-3′;
```

(SEQ ID NO: 16)

PNDEL,5′- TCT ggC ggT ggC CAT ATg gAC ATC gAg CTC
ACT CAg TCT CCA ACC ACC -3′;

(SEQ ID NO: 17)

EcoL,5′- CTC ggg AgA ATT CTA TCA TTT CAg CTC CAg
CTT ggT CCC ACA ACC gAA CgT gAg Cgg AAA gCT
gTg -3′.

The primers, STUVH and EcoL replaced Gly-44 in the V_{H} chain and Ala-100 in the V_{L} chain with cysteines, respectively (in boldface). These primers introduce restriction enzyme sites (underlined) for easy cloning of the V_{L} chain into Nde I-EcoRI site and of the V_{H} chain into Stu I-Hind III site in the expression vector.

Production of RiTs: 8H9(scFv)-PE3 8 or the two components of 8H9(dsFv)-PE38 (V_{L} and V_{H}-PE38) were expressed in E. coli, BL21(λDE3) and accumulated in inclusion bodies, as previously described (Brinkmann et al., Proc. Natl. Acad. Sci. USA 88: 8616-8620, 1991). Inclusion bodies were solubilized in Guanidine hydrochloride solution, reduced with dithioerythritol and refolded by dilution in a refolding buffer containing arginine to prevent aggregation, and oxidized and reduced glutathione to facilitate redox shuffling. Active monomeric protein was purified from the refolding solution by ion exchange and size exclusion chromatography (Onda et al., Cancer Res. 61: 5070-5077, 2001; Onda et al., J. Immunol. 163: 6072-6077, 1999). Protein concentration was determined by Bradford Assay (Coomasie Plus; Pierce, Rockford, Ill.). For the primate study a special batch of 8H9(dsFv)-PE38 was produced using precautions to remove endotoxin. The endotoxin content was less than 6 EU/mg.

Cytotoxicity Assay: The specific cytotoxicity of each IT was assessed by inhibition of protein synthesis by cells exposed to various concentrations of IT. Protein synthesis was measured as cellular incorporation of ³H-leucine (Brinkamn et al., Proc. Natl. Acad. Sci. USA 88: 8616-8620, 1991; Onda et al., J. Immunol. 163:6072-6077, 1999). Cells, at a concentration of (1.6×10⁴) cells/well, were plated in 96-well plates and incubated overnight. IT was diluted in PBS/0.2% BSA to desired concentrations and was added to the target cells in triplicate. The cells were incubated for 20 hours at 37° C., before the addition of 2 μCi ³H-leucine per well and further incubation for 2 hours at 37° C. Cells were frozen, thawed and harvested onto glass fiber filter mats using automated harvester. The radioactivity associated with the cells was counted in an automated scintillation counter. For competition experiments, excess 8H9 MAb or T6 MAb was added 15 minutes before the addition of the IT (15.5 ng/ml).

Toxicity in Mice: Groups of 5-10 female NIH Swiss mice were given single injections i.v. through the tail vein of escalating doses of ITs, as previously described (16). Animal mortality was observed over 2 wk. The LD₅₀ was calculated with the Trimmed Spearman-Karber program version 1.5, from the Ecological Monitoring Research Division, Environmental Monitoring Systems Laboratory, U.S. Environmental Protection Agency.

Monkey Studies: The monkey studies were performed under an approved protocol (LMB-045). For the toxicology studies one 9 kg monkey was injected with 8H9(dsFv)-PE38 0.1 mg/kg i.v. QOD×3 and the other 5 kg monkey with 0.2 mg/kg i.v. QOD×3. Plasma samples were obtained 10 minutes after each dose for blood level measurements and for blood chemistry measurements on days 1, 5, 8 and 15. To determine the blood levels of the RIT in monkeys, 200-400 times diluted plasma samples were incubated with MCF-7 cells overnight in cytotoxicity assay which is described in Cytotoxicity Assay section, and active immunotoxin quantitated by interpolation on a standard curve made from the cytotoxicity of purified immunotoxin (Onda et al., Cancer Res. 61: 5070-5077, 2001).

Anti-tumor Activity (In Vivo Anti-tumor Assay): The anti-tumor activity of RITs was determined in SCID mice bearing human cancer cells. MCF-7 cells (2×10⁶) were injected s.c. into SCID mice on day 0. Tumors (about 0.05 cm³ in size) developed in animals by day 4 after tumor implantation. Starting on day 4, animals were treated with i.v. injections of each of the RITs diluted in 0.2 ml of PBS/0.2% HSA. Therapy was given once every other day on days 4, 6, and 8; treatment groups consisted of 5 or 10 animals. Tumors were measured with a caliper every 2 or 3 days, and the volume of the tumor was calculated by using the formula: tumor volume (cm³)=length×(width)²×0.4. Two days before implanting MCF-7 cells, 17β-estradiol pellets (0.72 mg, 60 days release; Innovative Research of America, Sarasota, Fla.) were implanted s.c. because MCF-7 cells are estrogen dependent for growth. For the osteosarcoma model, 1.5×10⁶ OHS-M1 cells were planted s.c. without implanting 17β-estradiol pellets and treated using the identical protocol.

Statistical Analysis: Tumor sizes in animal experiments are expressed as mean±SD. For comparison between the two experimental groups, Mann-Whitney test was used. P<0.05 is considered statistically significant.

### Example 2

### Immunotoxin Construction

To determine whether the 8H9(scFv) could target a cytotoxic agent to antigen positive cells, two different RITs were constructed. Initially a single chain immunotoxin was made in which the Fv portion of MAb 8H9 is fused to PE38, a truncated form of PE. In the Fv, lysine at position 3 of the V_{H} is mutated to glutamine because glutamine is the most frequent amino acid in this position and the yields are often improved by this mutation (Onda et al., Cancer Res. 61: 5070-5077, 2001). Because the yield of the scFv immunotoxin was low (Table 1), a more stable disulfide linked immunotoxin (dsFv RIT) was constructed in which the light and heavy chains are linked by a stable disulfide bond.

TABLE 1
Yield and Activity of scFv and dsFv RIT

| | Yield of IT* | IC₅₀** |
|---|---|---|
| IT | (%) | (ng/ml) |
| 8H9(scFv)-PE38 | 1.7 | 5.0 ± 2 |
| 8H9(dsFv)-PE38 | 16 | 5.0 ± 2 |

*Yields refer to refolding yield (Buchner et al., Anal. Biochem. 205: 263-270, 1992).
**Cytotoxic activities were assessed on MCF-7 cells.

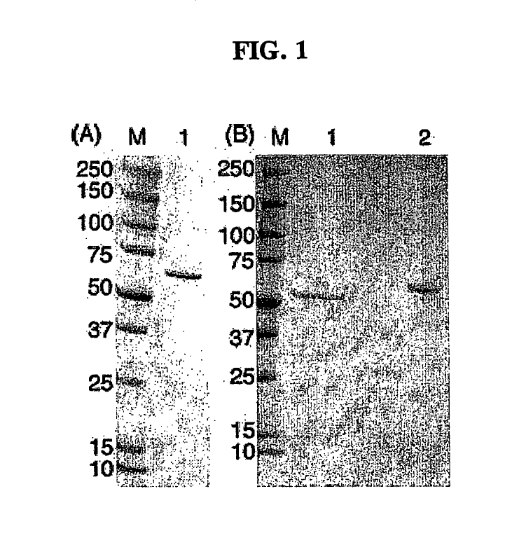

This procedure not only increases stability but often has the further advantage of increasing recombinant protein yield (Reiter et al., Nat. Biotechnol. 14:1239-12454, 1996). Both types of immunotoxins were produced in E. coli and purified by ion exchange and size exclusion chromatography after renaturation from inclusion bodies as previously described (Onda et al., J. Immunol. 163: 6072-6077, 1999). Each RIT eluted as a monomer upon TSK gel filtration chromatography and each migrated as a single band of about 62 kDa in SDS/PAGE (FIG. 1). Immunotoxin 8H9(scFv)-PE38 was prepared from a 1 liter culture of E. coli. After extensive washing 100 mg of inclusion body protein was recovered that was used to make immunotoxin. The final yield was 1.7 mg or 1.7%. In contrast 8H9(dsFv)-PE38 is prepared by combining inclusion body protein from cells grown separately that express the V_{L} protein or the VH-PE38 protein. When 33 mg of V_{L} protein was combined with 67 mg of V_{H}-PE38 protein, 16 mg of purified immunotoxin was recovered, or a 16% yield (Table 1) (Buchner et al., Anal. Biochem. 205: 263-270, 1992). Because of this high yield, the dsFv molecule was selected for further pre-clinical development. DNA encoding V_{L} protein and the V_{H}-PE³⁸ protein were deposit with the American Type Culture Collection (ATCC) in accordance with the Budapest treaty on Nov. 24, 2003.

### Example 3

### Cytotoxicity on Different Cell Lines

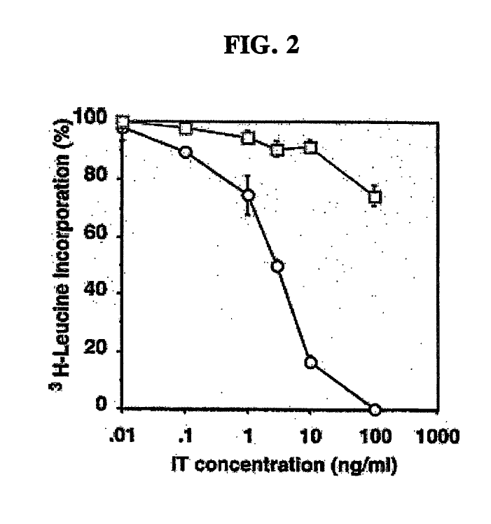

The ability of the 8H9(Fv)-PE38 to inhibit protein synthesis was used as a measure of its cytotoxic effect. A variety of antigen-positive cell lines and two antigen-negative cell lines were exposed to the RIT for 20 hours and ³H-leucine incorporation was then measured. MCF-7 cells, which react strongly with the 8H9 antibody, were the most sensitive to 8H9(scFv)-PE38 with an IC₅₀ of 5.0 ng/ml (FIG. 2, Table 2).

TABLE 2
Cytotoxic Activity of 8H9(Fv)-PE38 on Malignant Cell Lines

| | | IC₅₀ of | IC₅₀ of | |
|---|---|---|---|---|
| | | 8H9(Fv)- | M1(dsFv)- | 8H9 |
| | | PE38 | PE38* | reactivity |
| Original | Cell line | (ng/ml) | (ng/ml) | by FACS |
| Breast Ca | MCF-7 | 5.0 ± 2 | 300 | + |
| Breast Ca | BT-474 | 20.0 ± 0 | 900 | + |
| Breast Ca | ZR-75-1 | 35 ± 15 | >1000 | + |
| Osteosarcoma | U2OS | 30 ± 5 | >1000 | + |
| Osteosarcoma | CRL1427 | 50 ± 6 | >1000 | + |
| | (MG63) | | | |
| Osteosarcoma | OHS-M1 | 20 ± 2 | >1000 | + |
| Neuroblastoma | NMB-7 | 9.0 ± 1 | 300 | + |
| Neuroblastoma | LAN-1 | 12.5 ± 2 | 300 | + |
| Neuroblastoma | SK-N-BE(2) | 90 ± 8 | >1000 | + |
| Hodgkin's | L428 | >1000 | >1000 | − |
| Myeloma | SP2/0 | >1000 | >1000 | − |

*M1(dsFv)-PE38 is an IT against IL-2 receptor α subunit (Onda et al., 1991, supra; Onda et al., 1993, supra)

On two other breast cancer cell lines, BT-474 and ZR-75-1, which also react with MAb 8H9, the IC₅₀s were 20 and 35 ng/ml. Three osteosarcoma cell lines, U2OS, CRL1427, and OHS-M1, were also sensitive. The IC₅₀s were 30 ng/ml, 50 ng/ml, and 20 ng/ml, respectively. U2OS, CRL1427, and OHS-M1 react with MAb 8H9. Also three neuroblastoma cell lines, NMB-7, LA-N-1, and SK-N-BE(2) were sensitive to 8H9(Fv)-PE38 with IC₅₀s of 9.0 ng/ml, 12.5 ng/ml, and 90 ng/ml. On two cell lines that do not react with MAb 8H9, there was no cytotoxic effect at 1000 ng/ml.

After completing studies with the single chain immunotoxin the disulfide linked Fv molecule was prepared and tested on the MCF-7 cell line. The IC₅₀ of 8H9(dsFv)-PE38 is 5 ng/ml, which was similar to the cytotoxic activity of the single chain Fv molecule (Table 1).

### Example 4

### Cytotoxic Specificity

To determine whether the cytotoxic activity of 8H9(scFv)-PE38 is specific and requires binding to the antigen recognized by MAb 8H9, several control experiments were performed. The results in Table 2 shows that L428 and SP2/0 cells, which do not react with MAb 8H9, were not sensitive to 8H9(scFv)-PE38 (IC₅₀>1000 ng/ml). In addition, M1 (dsFv)-PE38, an immunotoxin that targets CD25, the a subunit of the IL-2 receptor, was not cytotoxic to cell lines killed by 8H9(scFv)-PE38.

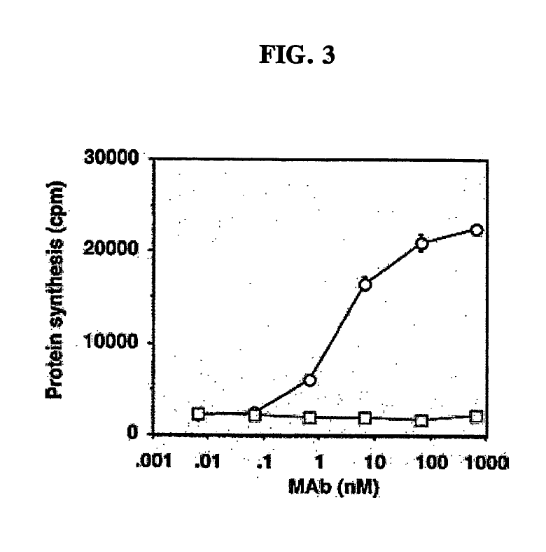

This specificity was confined in further work (FIG. 3). The cytotoxic activity of 8H9(scFv)-PE38 was competed by an excess amount of MAb 8H9 but not with MAb T6 that reacts with CD30 (Nagata et al., Clin. Cancer Res. 8: 2345-2355, 2002). In addition, MAb 8H9 alone (without the effector molecule) was not cytotoxic. Thus, specific binding to the 8H9 antigen and the toxic activity of PE38 are necessary for the cytotoxic activity of 8H9(Fv)-PE38.

### Example 5

### Nonspecific Toxicity in Mice

8H9(dsFv)-PE3 8 was evaluated for its nonspecific toxicity in mice. Groups of five or ten mice were injected i.v. once with varying doses of immunotoxin and observed for 2 weeks. Almost all of the deaths occurred within 72 hours after treatment. The mortality data is shown in Table 3. The LD₅₀ of 8H9(dsFv)-PE38 is 0.783 mg/kg (95% confidential range, 0.66-0.9295 mg/kg) calculated with the Trimmed Spearman-Karber Program version 1.

TABLE 3
Toxicity of 8H9(dsFv)-PE38 Administered to Mice i.v.

| Dose (mg/kg) | Mortality |
|---|---|
| 0.25 | 0/5 |
| 0.5 | 1/10 |
| 0.75 | 5/10 |
| 1.0 | 5/10 |
| 1.25 | 10/10 |
| 1.5 | 10/10 |

### Example 6

### Pilot Toxicology Study of 8H9(dsFv)-PE38 in Cynomolgus Monkey

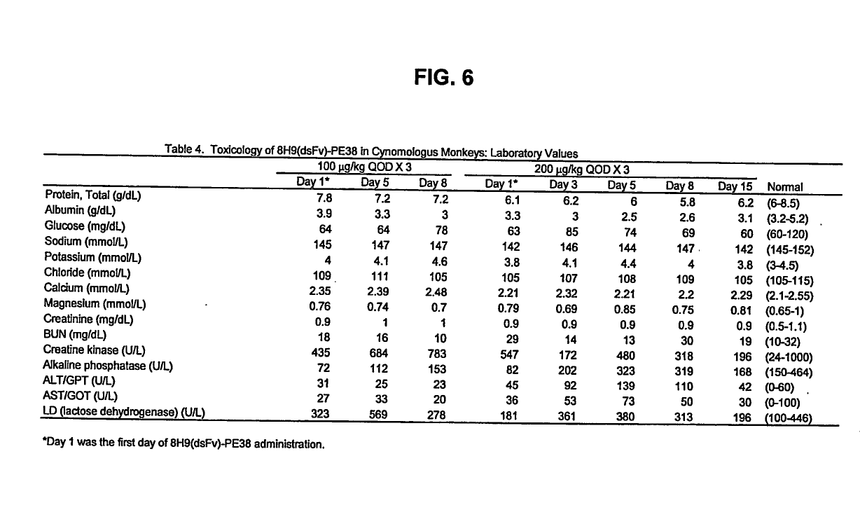

The toxicity of 8H9(dsFv)-PE38 was evaluated in two Cynomolgus monkeys. There is a similar reactivity of monkey and human tissues with 8H9 (Modak et al., Cancer Res. 61:4048-4054, 2001). One monkey received 0.1 mg/kg QOD×3 and the second received 0.2 mg/kg QOD×3. In this pilot study, both monkeys tolerated 8H9(dsFv)-PE38 well with only mild laboratory abnormalities (see FIG. 6 (Table 4)).

There was a slight decrease in albumin that was more pronounced in the high-dose monkey. The hepatic abnormalities were a borderline elevated ALT on days 3, 5, and 8 in the high-dose monkey, and the lactate dehydrogenase was borderline elevated on day 5 in the low-dose monkey. The major toxicity observed in both monkeys was loss of appetite. Thus, 8H9(dsFv)-PE38 could be administered safely to Cynomolgus monkeys, and the high-dose used (0.2 mg/kg) was higher than that needed to cause tumor regression of a human cancer xenograft in mice (0.15 mg/kg).

### Example 7

### Plasma Levels of 8H9(dsFv)-PE38 in Monkeys

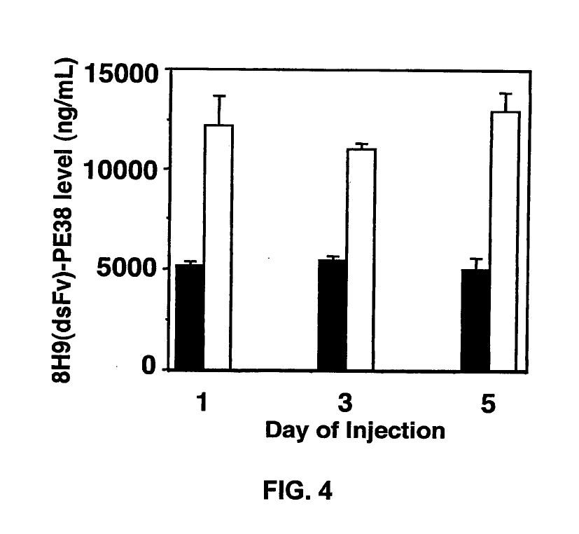

Serum levels of 8H9(dsFv)-PE38 were determined in each of the two monkeys ten minutes after each of the three doses. The levels were determined by cytotoxicity assay so that only intact cytotoxic protein would be measured. As shown in FIG. 4, the levels of 8H9(dsFv)-PE38 were 5.0-5.4 μg/ml 10 minutes after administration of 0.1 mg/kg and 11.0-13.0 μg/ml at 0.2 mg/kg. These blood levels are 1000-fold higher than the IC₅₀ of the immunotoxin on MCF-7 cells in cell culture.

Monkey studies can be useful in predicting toxicities, if the antibody reacts equally well with human and monkey tissues. Only two normal tissues from Cynomolgus monkeys also demonstrated a weak reactivity with nonspecific staining observed in stomach and liver (Modak et al., Cancer Res. 61: 4048-4054, 2001). To evaluate possible liver toxicity or other toxicities due to this cytoplasmic staining observed in immunohistochemical studies, a toxicology study was performed using two Cynomolgus monkeys. The injection of 0.1 mg/kg of 8H9(dsFv)-PE38 did not produce any increase in the level of liver enzymes in the blood of these monkeys and the injection of twice the dose produced only a small increase in liver enzymes. This data indicates that 8H9(dsFv)-PE38 has low toxicity for liver. It should also be noted that normal human brain tissue sections including frontal lobe, spinal cord, pons and cerebellum are completely negative for staining with 8H9 in immunohistochemical studies. Thus, 8H9(dsFv)-PE38 could potentially be administered in intrathecal therapy of leptomeningeal carcinomatosis from a wide spectrum of human solid tumors.

### Example 8

### Anti-tumor Activity in SCID Mice Bearing Human Cancer Cell Lines

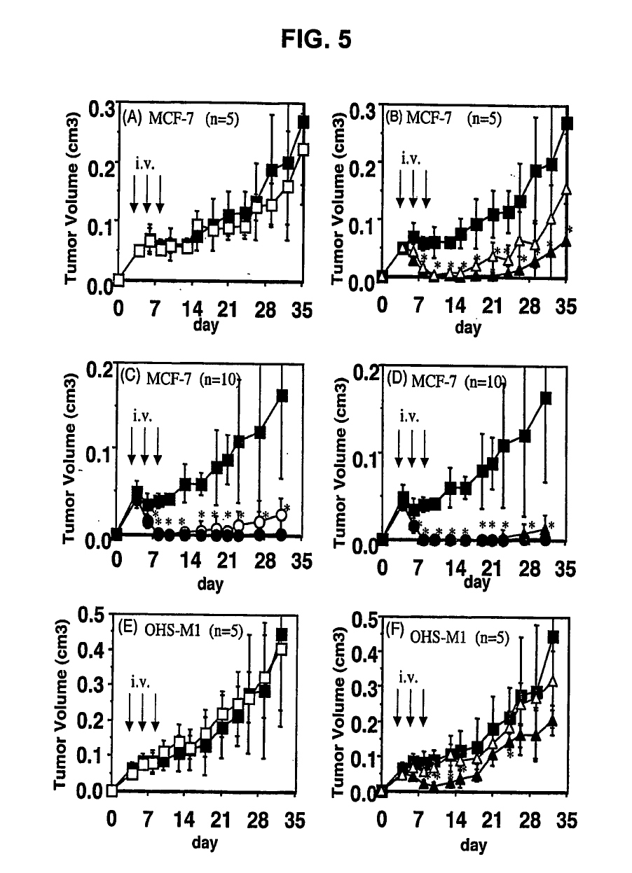

To determine the anti-tumor activity of 8H9(Fv)-PE38, several different doses of both the single chain and the disulfide linked Fv immunotoxin were administered to SCID mice bearing MCF-7 tumors or OHS-M1 tumors. The mice developed tumors about 50 mm³ in size by day 4 and were treated on days 4, 6, and 8. FIG. 5B shows tumor sizes in mice treated with 0, 0.075 or 0.15 mg/kg of 8H9(scFv)-PE38. In both groups of mice, tumor regressions were observed with the higher dose producing a larger effect. The control group received PBS/0.2% HSA. To determine whether anti-tumor activity was specific, mice were treated with a control immunotoxin that does not react with MCF-7 cells. M1(dsFv)-PE38, an IT directed at CD25, the a subunit of the IL-2 receptor (Onda et al., Cancer Res. 61:5070-5077, 2001) was chosen. Mice were injected with 0.15 mg/kg×3 of M1(dsFv)-PE38. No responses were noted with this treatment (FIG. 5A). M1(dsFv)-PE38 has previously been shown to produce complete regression of tumors expressing CD25 (Onda et al., Cancer Res. 61:5070-5077, 2001). To show the effects of 8H9(scFv)-PE38 were reproducible the anti-tumor experiments were carried a total of three times and observed specific tumor regressions in all of the experiments. In the second set of animal experiments the anti-tumor activity of 8H9(dsFv)-PE38 was evaluated at 0.075 and 0.15 mg/kg×3. Both doses were effective producing statistically significant and prolonged tumor regressions (FIG. 5C). In these experiments 8H9(scFv)-PE38 and 8H9(dsFv)-PE38 showed similar anti-tumor activities at 0.15 mg/kg (FIG. 5D).

The effects of 8H9(scFv)-PE38 was investigated on osteosarcoma cells. The OHS-M1 cell line forms tumors in SCID mice. Mice were injected with 1.5×10⁶ cells on day 0. The mice developed tumors about 50 mm³ in size by day 4 and were treated with immunotoxin i.v. on days 4, 6, and 8. FIGS. 5E and 5F show tumor sizes before and after treatment with 0.075 or 0.15 mg/kg of 8H9(Fv)-PE38. Although treatment with 0.075 mg/kg had little effect, tumor regressions were observed using 0.15 mg/kg. The average size of the tumors, which is indicated by (*), was statistically different between the control group and the immunotoxin injected group (P<0.05) for MCF-7 cells and for OHS-M1 cells. In comparison with the MCF-7 breast cancer tumors the osteosarcoma tumors are less responsive to 8H9(Fv)-PE38. This was consistent with the difference on IC₅₀s observed in cell culture experiments (MCF-7=5 ng/ml, OHS-M1=20 ng/ml). However, tumor regression of osteosarcomas was observed using the higher dose.

Thus, a single chain and a disulfide linked immunotoxin was prepared with the Fv portion of the 8H9 MAb. Both immunotoxins are specifically cytotoxic to cell lines reacting with the 8H9 antibody and both produce substantial tumor regressions in mice at doses that do not produce significant animal toxicity (FIG. 5). The 8H9 antibody was chosen for immunotoxin development, because it reacts with an antigen present on the cell surface of a variety of human cancers and does not appear to be expressed on the cell surface of normal tissues.

The 8H9 immunotoxins were tested against a panel of cell lines known to react with the 8H9 antibody. Many of these cell lines were killed by the immunotoxin. The most sensitive was the breast cancer cell line, MCF-7. When tested against MCF-7 tumors both immunotoxins produced substantial tumor regressions when given at 0.15 mg/kg. The immunotoxins could be of use in small volume disease after tumor reduction by surgery and chemotherapy.

The yield of the more stable disulfide linked immunotoxin molecule was much higher than the single chain molecule (16% compared to 1.7%, Table 1). The highest activity of 8H9(dsFv)-PE38 was observed on the MCF-7 cell line where the IC₅₀ is 5 ng/ml (0.8×10⁻¹⁰ M). One major factor determining the IC₅₀ is the affinity of the Fv for the target antigen. It is possible to increase the affinity and activity of other immunotoxins by 5-20-fold using site directed mutagenesis to alter amino acids in the complementarity determining regions (CDRs) of the Fvs (for example, see Salvatore et al., Clin. Cancer Res. 8: 995-1002, 2002).

In summary, two RITs, 8H9(scFv)-PE38 and 8H9(dsFv)-PE38 have been produced, which have a specific cytotoxic activity against cell lines derived from breast cancer, osteosarcoma, and neuroblastoma. Both immunotoxins showed specific anti-tumor activity using mouse xenograft models for human breast cancer and osteosarcoma. Cynomolgus monkeys tolerated the injection of this RIT without laboratory abnormalities.

It will be apparent that the precise details of the methods or compositions described may be varied or modified without departing from the spirit of the described invention. We claim all such modifications and variations that fall within the scope and spirit of the claims below.

1. An isolated Fv protein, comprising:
a) a variable region of a heavy chain of a monoclonal antibody that binds the antigen specifically bound by monoclonal antibody 8H9 and a variable region of a light chain of the monoclonal antibody that binds the antigen specifically bound by monoclonal antibody 8H9 wherein the variable region of a heavy chain of a monoclonal antibody that binds the antigen specifically bound by monoclonal antibody 8H9 and the variable region of a light chain of the monoclonal antibody that binds the antigen specifically bound by monoclonal antibody 8H9 are covalently linked by disulfide bonds; and
b) an effector molecule comprising a toxin;
wherein the Fv protein specifically binds the epitope bound by monoclonal antibody 8H9.

2. The isolated Fv protein of claim 1, wherein said effector molecule comprises ricin A, abrin, diphtheria toxin or a subunit thereof, Pseudomonas exotoxin or a portion thereof, saporin, restrictocin or gelonin.

3. The isolated Fv protein of claim 2, wherein said effector molecule is selected from the group consisting of PE38, PE40, PE38KDEL, and PE38REDL.

4. The isolated Fv protein of claim 1, wherein the variable region of the heavy chain comprises an amino acid sequence set forth as SEQ ID NO: 7, and wherein the variable region of the light chain comprises an amino acid sequence set forth as SEQ ID NO: 8.

5. (canceled)

6. The isolated Fv protein of claim 1, wherein the variable region of the heavy chain comprises
a heavy chain framework region comprising a complementarity determining region HCDR1, a HCDR2, and a HCDR3, wherein the (HCDR)-1 comprises an amino sequence NYDIN (amino acids 31-35 of SEQ ID NO: 3) the HCDR2 comprising an amino acid sequence WIFPGDGSTQY (amino acids 50-60 of SEQ ID NO: 3), the HCDR3 comprises an amino acid sequence QTTATWFAY (amino acids 99-107 of SEQ ID NO: 3).

7. The isolated Fv protein of claim 1, wherein the variable region of the light chain comprises
a light chain framework region comprising a complementarity determining region (LCDR)1, a LCDR2, and a LCDR3, wherein the LCDR1 comprises an amino acid sequence RASQSISDYLH (amino acids 157-167 of SEQ ID NO: 3), the LCDR2 comprises an amino acid sequence YASQSIS (amino acids 183-189 of SEQ ID NO: 3), and the LCDR3 comprises an amino acid sequence QNGHSFPLT (amino acids 222-230 of SEQ ID NO: 3).

8. The isolated Fv protein of claim 6, wherein the heavy chain framework and the light chain framework are human.

9. (canceled)

10. The isolated Fv protein of claim 9, wherein the toxin is covalently linked to the variable region of the heavy chain.

11. The isolated Fv protein of claim 10, wherein the toxin comprises a Pseudomonas exotoxin.

12. The isolated Fv protein of claim 11, wherein the Pseudomonas exotoxin is PE38.

13. The Fv of claim 1, wherein said Fv polypeptide comprises an amino acid sequence set forth as SEQ ID NO: 7 and an amino acid sequence set forth as SEQ ID NO: 8.

14-20. (canceled)

21. A pharmaceutical composition comprising a therapeutically effective amount of the isolated Fv protein of claim 1 sufficient to inhibit tumor cell growth, and a pharmaceutically acceptable carrier.

22. The composition of claim 21, wherein said effector molecule is a Pseudomonas exotoxin.

23. The composition of claim 21, wherein the Pseudomonas exotoxin molecule comprises PE38, PE40, PE38KDEL or PE38REDL.

24. A method for killing a tumor cell, comprising contacting the cell with an effective amount of the isolated Fv protein of claim 1, thereby killing the cell.

25. The method of claim 24, wherein the cell is in vitro.

26. The method of claim 24, wherein the cell is in vivo.

27. The method of claim 24, wherein the Fv protein comprises
an effector molecule comprising ricin A, abrin, diphtheria toxin or a subunit thereof, Pseudomonas exotoxin or a portion thereof, saporin, restrictocin or gelonin.

28. The method of claim 27, wherein the effector molecule comprises a Pseudomonas exotoxin.

29. The method of claim 28, wherein the Pseudomonas exotoxin comprises PE35, PE37, PE38 or PE40.

30. The method of claim 29, wherein the Pseudomonas exotoxin is PE38.

31. The method of claim 24, wherein the cell is a breast cancer cell, an osteosarcoma cell, or a neuroblastoma cell.

32. A method for treating a tumor in a subject, comprising administering to the subject a therapeutically effective amount of the Fv protein of claim 1, thereby treating the tumor.

33. The method of claim 32, wherein the tumor is a breast cancer, an osteosarcoma, or a neuroblastoma.

34-38. (canceled)
